(19) United States
(12) Patent Application Publication
(10) Pub. No.: US 2023/0316208 A1
(43) Pub. Date: Oct. 5, 2023

(54) SYSTEMS AND METHODS FOR MAINTAINING HEALTHY ENVIRONMENTS

(71) Applicant: Anthony Mancillas, Los Angeles, CA (US)

(72) Inventor: Anthony Mancillas, Los Angeles, CA (US)

(21) Appl. No.: 18/127,874

(22) Filed: Mar. 29, 2023

Related U.S. Application Data
(60) Provisional application No. 63/324,735, filed on Mar. 29, 2022.

(51) Int. Cl. G06Q 10/0639; A47K 5/12; A61L 2/10

(52) U.S. Cl. CPC G06Q 10/06398; A47K 5/1217; A61L 2/10

(57) ABSTRACT

A system and method for maintaining healthy environments that includes a sensor and a non-transitory memory storing an executable code and a hardware processor executing the executable code to receive an input from the sensor, determine an occurrence of an event, and update a station profile based on a signal that includes a station information. Also included is a fluid reservoir storing a fluid and a fluid dispensing mechanism fluidically connected to the fluid reservoir to dispense the fluid when operated by a user. The non-transitory memory additionally stores a database including a plurality of user profiles and the system and method further includes receiving an identification signal from a scanner device and updating a user profile of the plurality of user profiles based on the identification signal. The system and method may be further configured to determine a compliance and formulate strategy based on determination of the compliance.

### BACKGROUND

Presently, in the interest of promoting health and hygiene in the workplace and public spaces, automatic dispensing devices are used to dispense a variety of liquids, such as hand sanitizer and hand soap. Despite having such devices available, some workplace facilities do not have such devices readily available for use. Meanwhile, in the workplace facilities that do have such devices readily available to use, there is a general lack of compliance with simple practices of hand washing and sanitizing. Additionally, while some employees take sick leave, many employees choose to go into work even while sick, further spreading illness throughout the workplace facilities. Due to poor health and hygiene, employers incur significant associated costs for worker illnesses. There remains a need to improve compliance and usage of such automatic dispensing devices. The present disclosure provides for a novel system and methods for maintaining healthy environments that address the above noted problems in efforts to promote workforce health and facility safety, improve compliance, reduce absenteeism due to illness and spread of infections and diseases, reduce sick leave costs, obtain better rates for group health insurance coverage, overall sustainability, and the like.

### SUMMARY

The present disclosure is directed to systems and methods for maintaining healthy environments, substantially as shown in and/or described in connection with at least one of the figures, as set forth more completely in the claims.

In some implementations, the monitoring system for use with an automatic dispensing device includes at least one sensor, and a computing device, wherein the computing device includes a non-transitory memory storing an executable code and a hardware processor executing the executable code to receive an input from the at least one sensor, the input including a vibration information, determine an occurrence of an event, and update a station profile based on a signal that includes a station information.

In some implementations, the monitoring system further includes a fluid reservoir storing a fluid for dispensing and a fluid dispensing mechanism fluidically connected to the fluid reservoir, the automatic dispensing device configured to dispense a user portion of the fluid when operated by a user, wherein the user portion of the fluid is a calibrated volume.

In some implementations of the monitoring system, the at least one sensor includes one or more of an accelerometer, a gyroscope, a magnetometer, a radiofrequency sensor, a vibration sensor, a motion sensor, a biometric sensor, a temperature sensor, a proximity sensor, an air quality sensor, a volatile organic compound (VOC) sensor, and an image sensor.

In some implementations of the monitoring system, the station profile includes at least one of a refill identifier, a refill volume, a fill level information, a viscosity information, a date of last refill, a number of operations since last refill, a frequency of use information, a user counter, a battery level, and an air quality information.

In some implementations of the monitoring system, the non-transitory memory additionally stores a database including a plurality of user profiles and the monitoring system further comprises a scanner device configured to receive an identification signal from the scanner device and update a first user profile of the plurality of user profiles based on the identification signal.

In some implementations of the monitoring system, the user profile includes at least one of an individual identification, a time tracking information, a temperature reading, and a usage counter.

In some implementations, the monitoring system is further configured to determine a compliance and formulate a strategy based on the determination of the compliance, wherein the strategy is implementing at least one of a training plan, a marketing plan, an incentive plan, and an advertisement program.

In some implementations, the monitoring system is at least one of a battery-powered and a solar-powered.

In some implementations, the monitoring system further includes an ultraviolet (UV) light sanitizer for sanitizing a personal object.

In some implementations of the monitoring system, the event is one of an operation of the automatic dispensing device and an individual walking past the automatic dispensing device.

In another implementation, a method for use with a monitoring system includes a non-transitory memory and a hardware processor, wherein the method includes receiving, using the hardware processor, an input from at least one sensor, the input including a vibration information, determining, using the hardware processor, an occurrence of an event, and updating, using the hardware processor, a station profile based on a signal that includes a station information.

In some implementations of the method, the monitoring system monitors an automatic dispensing device having a fluid reservoir that stores a fluid for dispensing and a fluid dispensing mechanism fluidically connected to the fluid reservoir, wherein the automatic dispensing device is configured to dispense a user portion of the fluid, wherein the user portion of the fluid is a calibrated volume, and wherein the method further comprises dispensing a user portion when operated by a user.

In some implementations of the method, the at least one sensor includes one or more of an accelerometer, a gyroscope, a magnetometer, a radiofrequency sensor, a vibration sensor, a motion sensor, a biometric sensor, a temperature sensor, a proximity sensor, an air quality sensor, a VOC sensor, and an image sensor.

In some implementations of the method, the station profile includes at least one of a refill identifier, a refill volume, a fill level information, a viscosity information, a date of last refill, a number of operations since last refill, a frequency of use information, a user counter, a battery level, and an air quality information.

In some implementations of the method, the non-transitory memory additionally stores a database including a plurality of user profiles wherein the method further comprises receiving an identification signal from a scanner device and updating a first user profile of the plurality of user profiles based on the identification signal.

In some implementations of the method, the user profile includes at least one of an individual identification, a time tracking information, a temperature reading, and a usage counter.

In some implementations of the method, the method further comprises determining a compliance and formulating a strategy based on the determination of the compliance, wherein the strategy is implementing at least one of a training plan, a marketing plan, an incentive plan, and an advertisement program.

In some implementations of the method, the monitoring system is at least one of a battery-powered and a solar-powered.

In some implementations of the method, the monitoring system further includes an ultraviolet (UV) light sanitizer for sanitizing a personal object.

In some implementations of the method, the event is one of an operation of an automatic dispensing device and an individual walking past the automatic dispensing device.

### BRIEF DESCRIPTION OF THE DRAWINGS

FIG. 1 shows a diagram of an exemplary system for maintaining healthy environments, according to one implementation of the present disclosure;

FIG. 2 shows a flowchart illustrating an exemplary method of maintaining healthy environments, according to one implementation of the present disclosure; and

FIG. 3 shows a flowchart illustrating an exemplary method of maintaining healthy environments, according to one implementation of the present disclosure.

### DETAILED DESCRIPTION

The following description contains specific information pertaining to implementations in the present disclosure. The drawings in the present application and their accompanying detailed description are directed to merely exemplary implementations. Unless noted otherwise, like or corresponding elements among the figures may be indicated by like or corresponding reference numerals. Moreover, the drawings and illustrations in the present application are generally not to scale, and are not intended to correspond to actual relative dimensions.

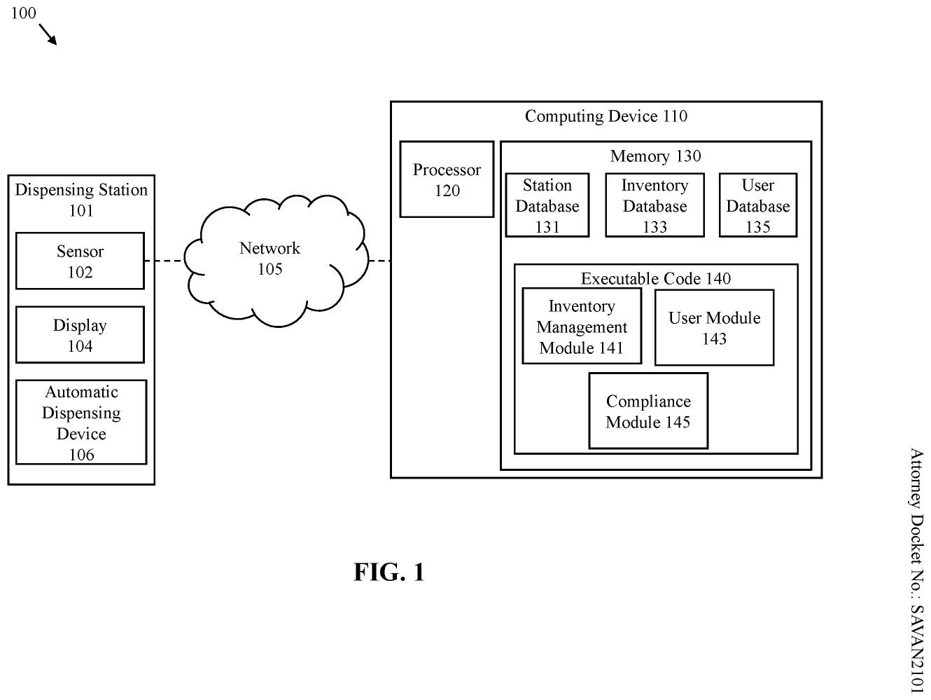

FIG. 1 shows a diagram of an exemplary system for maintaining healthy environments, according to one implementation of the present disclosure. System 100 includes dispensing station 101, network 105, and computing device 110. Dispensing station 101 includes automatic dispensing device 106. Automatic dispensing device 106 includes a fluid reservoir for storing fluids or liquids, such as hand sanitizer, soap, and the like. Further, automatic dispensing device 106 includes a fluid dispensing mechanism fluidically connected to the fluid reservoir. In some implementations, automatic dispensing device 106 may dispense other types of fluids or liquids, including alcohols, air freshener, cleaning detergents, laundry detergents, fabric softeners, and the like. In the depicted implementation, dispensing station 101 may include sensor 102 and display 104. In some implementations, system 100 may display visual images and videos on display 104. In some implementations, visual images may include advertisements, marketing materials, user profiles, workplace information, and more. In some implementations, display 104 may be an integrated display in dispensing station 101. In other implementations, display 104 may be attached to dispensing station 101. In some implementations, display 104 may be communicatively connected to computing device 110, either by a wired connection or a wireless connection. In some implementations, display 104 may be a television display, a computer display, a mobile telephone display, a tablet computer display, or other technology capable of displaying or conveying images and/or video. In some implementations, system 100 may be a substantially touchless system in the course of ordinary daily usage.

In the depicted implementation, dispensing station 101 includes sensor 102. In some implementations, dispensing station 101 includes at least one sensor 102. In other implementations, sensor 102 may be a standalone sensor for placing at a location to be monitored in an environment. Appropriate locations for placing sensor 102 include locations where a detectable environmental substance may be measured. By way of non-limiting example, locations may include entry and exit doorways, doorways leading to rooms, such as meeting rooms or restrooms, windows, heating, ventilation, and air conditioning (HVAC) vents, and hand sanitizing stations. In some implementations, sensor 102 is one sensor. In some implementations, system 100 may include more than one sensor 102. In some implementations, one sensor 102 includes one or more of an accelerometer, a gyroscope, a magnetometer, a radiofrequency sensor, a vibration sensor, a motion sensor, a volume sensor, a biometric sensor, a temperature sensor, a proximity sensor, an air quality sensor, a volatile organic compound (VOC) sensor, an image sensor, a pressure sensor, and a liquid level sensor. Various sensor types may be used in combination with each other.

In some implementations, an accelerometer, a gyroscope, a magnetometer, and any combination thereof may be included for detecting motion and orientation. In some implementations, an accelerometer can sense motion and is used to measure acceleration, detect movement, shock, or vibration of automatic dispensing device 106. In some implementations, gyroscopes are used to measure rotational motion of automatic dispensing device 106. In some implementations, a magnetometer may be used to detect motion and orientation of automatic dispensing device 106 of dispensing station 101, wherein the magnetometer detects changes in magnetic field caused by the movement of magnetized objects or parts of an activated electric motor of automatic dispensing device 106.

In some implementations, the magnetometer may detect the state of the battery charge by measuring the magnetic field around battery cells. The magnetometer may detect magnetic flux when the electric motor of automatic dispensing device 106 is activated. The change in magnetic field when the electric motor is activated or deactivated results in a change in the magnetic field which may be detected by the magnetometer. In some implementations, the magnetometer may be calibrated to assess the charge of the battery in a battery powered station. For example, the magnetic flux may be greater when the battery is fully charged. As the battery charge diminishes, the magnetic flux when the electric motor starts or stops may be reduced. In some implementations, the reduced magnetic flux is used to determine the low-charge status of the battery. In some implementations, any combination of two or more of an accelerometer, a gyroscope, and a magnetometer may provide for greater accuracy in detecting motion and orientation compared to having one of the three sensors alone.

In some implementations, an air quality sensor measures smoke, dust, other particulates, pollutants, noxious gases, and other contaminants in the air. In some implementations, a VOC sensor directly measures ambient concentrations of a broad range of gases associated with bad air quality. In some implementations, a VOC sensor is integrated into an air treatment device and/or air quality monitor. In some implementations, a VOC sensor is included at or near dispensing station 101. In some implementations, one or more of an air quality sensor and a VOC sensor may be placed at or near doors and windows. In some implementations, one or more of an air quality sensor and a VOC sensor may be placed at or near HVAC vents. The location placement of a VOC sensor may detect and monitor VOC levels and gas leaks within the vicinity of its placement. In some implementations, the placement of the VOC sensor may further indicate the location of a gas leak as well.

In some implementations, automatic dispensing device 106 and sensor 102 are a kit. In some implementations, sensor 102 is attached to dispensing station 101 on or near automatic dispensing device 106. In some implementations, sensor 102 is communicatively connected to computing device 110. In some implementations, sensor 102 is directly connected to computing device 110. In some implementations, the connection between sensor 102 and computing device 110 may be a wired connection. In some implementations, the connection between sensor 102 and computing device 110 may be a wireless connection. In the depicted implementation of FIG. 1, sensor 102 may be further connected to computing device 110 through network 105. Network 105 is a computer network. In some implementations, network 105 may be a local area network, a wide area network, a wired network, a wireless network, or the internet. In some implementations, network 105 may be a cellular network or a mobile network.

Computing device 110 is a computing system for monitoring automatic dispensing device 106. As shown in FIG. 1, computing device 110 includes processor 120 and memory 130. Processor 120 is a hardware processor, such as a central processing unit (CPU) found in computing devices. Memory 130 is a non-transitory storage device for storing computer code for execution by processor 120, and also for storing various data and parameters. As depicted in FIG. 1, memory 130 includes station database 131, inventory database 133, user database 135, and executable code 140. In some implementations, computing device 110 is a central node in a computer network. Dispensing station 101 may be a node connected to the computer network. Sensor 102 may be a node connected to the computer network. System 100 may include a plurality of dispensing stations, a plurality of sensors, and a plurality of nodes connected to computing device 110. In some implementations, system 100 may be connected to the internet or another computer network through computing device 110.

Executable code 140 is a computer algorithm stored in memory 130 for execution by processor 120 to monitor automatic dispensing device 106. Executable code 140 may include one or more software modules for execution by processor 120. In the depicted implementation, executable code 140 includes inventory management module 141, user module 143, and compliance module 145. In some implementations, executable code 140 may include more than three software modules for execution by processor 120. In some implementations, executable code 140 may include less than three software modules for execution by processor 120. In some implementations, system 100 may be a cloud-based and/or open-source software platform for monitoring automatic dispensing device 106 configured for web, mobile, and desktop devices.

In some implementations, station database 131 may be a database storing information related to dispensing station 101, including location information, make and model of automatic dispensing device 106, and other related information. In some implementations inventory database 133 may be a database storing information related to automatic dispensing device 106, including metrics for automatic dispensing device 106, refill inventory compatible with automatic dispensing device 106, and other related information. In some implementations, user database 135 may be a database storing information related to a plurality of user profiles.

Inventory management module 141 is a software module stored in memory 130 for execution by processor 120 to manage inventory of dispensing station 101, according to one implementation of the present disclosure. In some implementations, inventory management module 141 may receive an input from at least one sensor 102, the input including a vibration information. In some implementations, inventory management module 141 may determine occurrence of operation of automatic dispensing device 106. In some implementations, inventory management module 141 may calculate the number of operations of automatic dispensing device 106 since last refill. In some implementations, inventory management module 141 may determine if a fluid reservoir level has reached the threshold to order refill for automatic dispensing device 106. In some implementations, inventory management module 141 may implement protocol to order refill for automatic dispensing device 106. In some implementations, inventory management module 141 may update dispensing station 101 profile.

User module 143 is a software module stored in memory 130 for execution by processor 120 to provide user profile information, according to one implementation of the present disclosure. In some implementations, user module 143 may receive an identification signal from a scanner device. In some implementations, user module 143 may update a user profile of a plurality of user profiles based on the identification signal. In some implementations, user module 143 may update a user profile of a plurality of user profiles based on one or more signals received from sensor 102.

Compliance module 145 is a software module stored in memory 130 for execution by processor 120 to ascertain a compliance with health and safety practices, according to one implementation of the present disclosure. In some implementations, compliance module 145 may determine a compliance. In some implementations, compliance module 145 may formulate a strategy based on determination of the compliance.

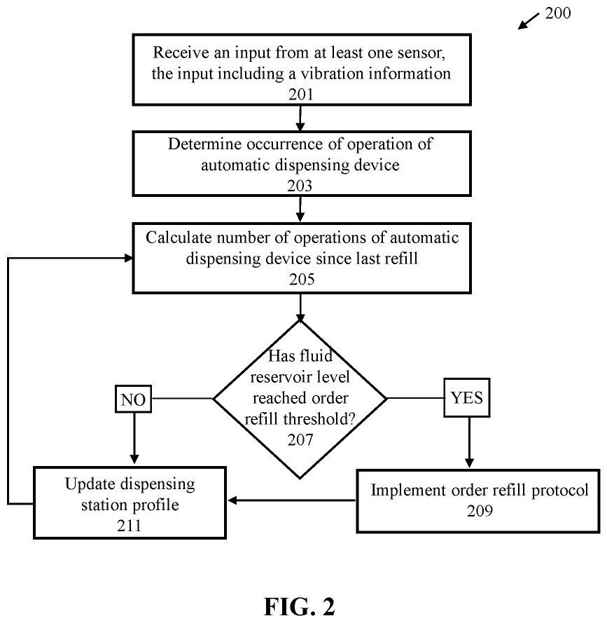

FIG. 2 shows a flowchart illustrating an exemplary method of maintaining healthy environments, according to one implementation of the present disclosure. Method 200 begins at 201, with inventory management module 141, using hardware processor 120, receiving an input from at least one sensor 102, the input including a vibration information. In some implementations, one sensor 102 includes one or more of an accelerometer, a gyroscope, a magnetometer, a radiofrequency sensor, a vibration sensor, a motion sensor, a biometric sensor, a temperature sensor, a proximity sensor, an air quality sensor, a VOC sensor, and an image sensor. In some implementations, input from at least one sensor 102 may provide information to monitor and track use of dispensing station 101. In some implementations, the vibration information may inform computing device 110 of the occurrence of an event. In some implementations, the event may be one of an operation of automatic dispensing device 106 and an individual walking past automatic dispensing device 106. In some implementations, dispensing station 101 may include a global positioning system (GPS) to provide the location of dispensing station 101.

Method 200 continues at 203, with inventory management module 141, using hardware processor 120, determining the occurrence of an operation of automatic dispensing device 106. Automatic dispensing device 106 may have a fluid reservoir that has a volume for storing an amount of fluid or liquid for dispensing, wherein the fluid may be hand sanitizer, soap, and the like. Additionally, automatic dispensing device 106 may further have a fluid dispensing mechanism fluidically connected to the fluid reservoir, wherein automatic dispensing device 106 is configured to dispense a user portion of the fluid stored in the fluid reservoir. In some implementations, the fluid dispensing mechanism is fluidically connected to the fluid reservoir, such that the fluid dispensing mechanism is configured to dispense a user portion of the fluid stored in the fluid reservoir when automatic dispensing device 106 is operated by a user. In some implementations, the user portion of the fluid is a calibrated volume. In some implementations, the operation of automatic dispensing device 106 includes dispensing a user portion of the fluid by the fluid dispensing mechanism when operated by the user. In some implementations, present disclosure contemplates monitoring real-time operation of automatic dispensing device 106 and calibrates accordingly, taking into account fluid viscosity, reservoir size, and other information in dispensing station profile. In some implementations, other types of fluids or liquids for dispensing include alcohols, air freshener, cleaning detergents, laundry detergents, fabric softeners, and the like.

In some implementations, the present disclosure contemplates automatic dispensing device 106 may also dispense a volume of fluid to refill a personal sanitizer container. In some implementations, when refilling a personal sanitizer container, monitoring system may detect when the container reached a full threshold and automatically stop dispensing the fluid for refill. In some implementations, the present disclosure contemplates automatic dispensing device 106 may also dispense or spray air freshener. In some implementations, an input from an air quality sensor including an air quality information may cause an occurrence of operation of automatic dispensing device 106 to dispense or spray air freshener.

In some implementations, a vibration information may inform whether the event is the operation of automatic dispensing device 106 dispensing the fluid. In some implementations, a vibration information may inform whether the event is not the operation of automatic dispensing device 106 dispensing the fluid. In some implementations, the present disclosure contemplates incorporating machine learning capabilities into the monitoring system. In some implementations, machine learning may be incorporated to recognize a vibration information corresponding to a dispensing event in different environments. For example, machine learning may be desired in high-vibration environments, such as manufacturing and research facilities, to distinguish between a baseline vibration information of the environment and a vibration information of operation of automatic dispensing device 106. In some implementations, the present disclosure contemplates further machine learning capabilities including recognizing human and non-human motion, biometric identification, temperatures, images, proximities, fluid viscosities, air quality, liquid level, and more.

Method 200 continues at 205, with inventory management module 141, using hardware processor 120, calculating the number of operations of automatic dispensing device 106 since last refill. In some implementations, inventory database 133 may include a database of automatic dispensing device 106, including metrics for automatic dispensing device 106 from different manufacturers and brands. In some implementations, metrics for automatic dispensing device 106 may include respective fluid reservoir size, number of operations of automatic dispensing device 106 since last refill, date of last refill, and other measurements. In some implementations, the calculated number of operations of automatic dispensing device 106 since last refill may be compared with metrics information in inventory database 133 to inform fluid reservoir fill level information.

Method 200 continues at 207, with inventory management module 141, using hardware processor 120, determining if a fluid reservoir level has reached the threshold to order refill for automatic dispensing device 106. In some implementations, the calculated number of operations of automatic dispensing device 106 since last refill may inform when fluid reservoir level has reached the threshold to order refill. In some implementations, inventory database 133 may include information on the threshold fill level in the fluid reservoir to order refill for automatic dispensing device 106. In some implementations, inventory database 133 has information on the number of operations of automatic dispensing device 106 since last refill to reach the order refill threshold fill level in the fluid reservoir. In some implementations, the present disclosure contemplates the detected vibrations from operation of automatic dispensing device 106 may also inform a fill level of the fluid reservoir. For example, in some implementations, a greater detected vibration may indicate that refill is running low as the fluid reservoir may have more empty, hollow space; conversely, in some implementations, a lower detected vibration may indicate the fluid reservoir is still full. In some implementations, a liquid level sensor may detect a liquid level or a fill level in the fluid reservoir.

In some implementations, inventory database 133 may include information on different types of refills compatible with automatic dispensing device 106 at dispensing station 101. Various types of liquid refills of different viscosities may include liquid soap, foaming soap, gel sanitizer, foam sanitizer, liquid sanitizer, detergent, air freshener, and more. In some implementations, refills may span different genres, including various laundry-related liquids such as liquid laundry detergent, liquid fabric softener, liquid bleach, air freshener, and more. In some implementations, types of refills may range from rubbing alcohol to vodka and more. In some implementations, automatic dispensing device 106 may include more than one spout. In some implementations, each spout may be optimized to dispense fluid of a specific viscosity. In some implementations, each spout may be optimized to dispense fluids of different viscosities. In some implementations, a spout may spray fluid, such as an air freshener. In some implementations, a spout may spray an aerosol, such as an air freshener.

Method 200 continues at 209, such that if the fluid reservoir level has reached the order refill threshold, inventory management module 141, using hardware processor 120, implements a protocol to order a refill for automatic dispensing device 106. In some implementations, when order refill threshold is reached, automatic reorder of the refill inventory is placed. In some implementations, an automatic alert will notify a refill is needed. In some implementations, refilling automatic dispensing device 106 may entail manually filling or pouring the fluid reservoir with the proper fluid. In some implementations, refilling automatic dispensing device 106 may entail replacing an empty cartridge with a refill cartridge. The present disclosure also contemplates a bottle refill service, wherein the service stops at each dispensing station 101, cleans out bottles, refills the fluid reservoir, and continues onto the next station. In some implementations, bottle refill service may be completed by a mobile van service.

Method 200 continues at 211, in some implementations, with inventory management module 141, using hardware processor 120, updating the dispensing station profile in inventory database 133. In some implementations, inventory management module 141, using hardware processor 120, may update a station profile based on a signal that includes a station information. In some implementations, after implementing order refill protocol 209, inventory management module 141, using hardware processor 120, updates dispensing station profile accordingly as shown at 211. Additionally, in some implementations, if the fluid reservoir level has not reached the order refill threshold, inventory management module 141, using hardware processor 120, updates dispensing station profile accordingly as shown at 211. In some embodiments, the dispensing station profile includes at least one of a refill identifier, a refill volume, a fill level information, a viscosity information, a date of last refill, a number of operations since last refill, a frequency of use information, a user counter, a battery level, and an air quality information. In some embodiments, a refill identifier may include information about the refill, e.g., brand or manufacturer of refill, soap, hand sanitizer, detergent, etc. In some implementations, monitoring system calibrates based on information included in dispensing station profile. In some embodiments, a refill volume may be the volume of the refill. In some embodiments, a fill level information may include amount or level of fluid in the fluid reservoir. In some embodiments, a viscosity information may inform whether the refill may be foam, liquid, gel, or another type of fluid. In some embodiments, a date of last refill is the most recent date on which the device was refilled. In some embodiments, a number of operations since last refill is a tally or counter of operations of automatic dispensing device 106 since the most recent refill.

In some embodiments, a frequency of use information may include calculations of the number of operations of automatic dispensing device 106 within a specified period of time, the number of operations of automatic dispensing device 106 divided by the number of times an individual walks past automatic dispensing device 106, and other calculations of rates of occurrence. In some embodiments, a user counter may be the number of individuals that use the dispensing device at dispensing station 101 by operating automatic dispensing device 106.

In some embodiments, a battery level of the monitoring system for use with automatic dispensing device 106 indicates the amount of battery power level, which may inform time to charge batteries or change batteries. In some embodiments, the system is at least one of a battery-powered and a solar-powered. The present disclosure further contemplates that dispensing station 101 may further include an air quality sensor, and therefore the dispensing station profile may also include air quality information. In some implementations, an air quality sensor or air quality monitor may be connected to computing device 110 through a wireless network. In some implementations, an air quality sensor or air quality monitor may be connected to computing device 110 through a cellular network.

In some implementations, once 211 has completed updating dispensing station profile, the method returns to 205 to calculate the number of operations and proceed through the steps of method 200 again.

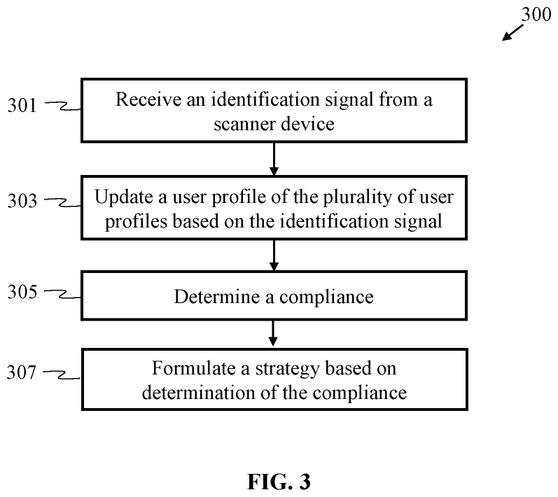

FIG. 3 shows a flowchart illustrating an exemplary method of maintaining healthy environments, according to one implementation of the present disclosure. In some implementations, the method for of maintaining healthy environments may include a method for monitoring automatic dispensing devices. Method 300 begins at 301, with user module 143, using hardware processor 120, receiving an identification signal from a scanner device. In some implementations, scanner device may be attached to dispensing station 101. In some implementations, scanner device may be built-in with dispensing station 101. In some implementations, scanner device may scan an identification badge or card or another form of identification. In some implementations, a scanner device may include a proximity sensor to detect a presence and/or proximity of individuals and/or potential users. In some implementations, a proximity sensor may detect a presence and/or proximity of potential users while device is in a dormant state. In some implementations, dispensing station 101 may include one or more biometric sensors to verify and/or recognize the identity of potential users. In some implementations, biometric sensors may be further linked to a building system serving various functions, including security purposes, entrance and exit control, and more. In some implementations, biometrics may include one or more of a fingerprint scan, a voice recognition, a speech recognition, a facial recognition, an iris recognition, a retinal scan, a heart-rate sensor, a hand geometry recognition, and other types of biometric techniques. In some implementations, scanner device or card reader is touchless that can be used for payment and recording entry and/or egress. In some implementations, the present disclosure contemplates the monitoring system includes a code reader, e.g., QR code scanner.

In some implementations, user database 135 may include a plurality of user profiles. In some implementations, user profiles in database 135 may be user profiles of employees, facilities management workers, and other individuals who have access to the facility in which the dispensing station 101 is located. In some implementations, user database 135 may include information to recognize regular users and distinguish from guest users. In some implementations, guest users may use a guest identification badge or card. In some implementations, dispensing station 101 may be linked to the building or facility system, for employees and workers to clock in and clock out.

Method 300 continues at 303, with user module 143, using hardware processor 120, updating a user profile of the plurality of user profiles based on the identification signal. In some implementations, a user profile includes at least one of an individual identification, a time tracking information, a temperature reading, and a usage counter. In some implementations, individual identification identifies the user of the dispensing station 101. In some implementations, user profile includes information for tracking an employee's time for clocking in and clocking out of work. In some implementations, dispensing station 101 may include a temperature sensor, which may measure an individual's body temperature, such that user profile may include a temperature reading. In some implementations, the system also tracks the user's usage or operation of automatic dispensing device 106 to disinfect the user's hands for example. In some implementations, a user's usage may include a usage counter of the number of times the user has operated automatic dispensing device 106 within a period of time (e.g., daily, weekly, monthly, annually, and/or any other designated period of time). In some implementations, a user's usage may include a user's frequency of use information. In some implementations, such information in the user profile may inform attendance, time tracking, temperature reading, and may provide further instructions to sanitize if necessary. The present disclosure also contemplates dispensing station 101 further comprising an image sensor to check that an individual is wearing personal protective equipment such as a face mask if one is required.

Method 300 continues at 305, with compliance module 145, using hardware processor 120, determining a compliance. In some implementations, compliance may include abiding by business, building or facility recommendations and/or regulations in efforts to promote health and safety practices. For example, facilities may encourage regular practice of handwashing and hand sanitizing, thus a compliance may include user operation of automatic dispensing device 106 to disinfect hands and/or to wash hands. In some implementations, determining a compliance may include analytics and/or calculations based on data compiled from one or more of a station profile, a plurality of station profiles, a user profile, and a plurality of user profiles. For example, determining a compliance for dispensing station 101 may include calculating a percentage of individuals who activate the operation of automatic dispensing device 106 when walking by dispensing station 101. In some implementations, determining a compliance for dispensing station 101 may include tallying users or individuals within an area or zone of the building or facility that comply with facility recommendations. In some implementations, location information of dispensing station 101 may inform compliance. In some implementations, a building with more than one floor level may use analytics in determining a compliance for each floor level.

Method 300 continues at 307, with compliance module 145, using hardware processor 120, formulating a strategy based on determination of the compliance. In some implementations, strategy may include implementing at least one of a training plan, a marketing plan, an incentive plan, and an advertisement program. In some implementations, the training plan may vary depending on the level or percentage of compliance. In some implementations, where user compliance falls below a target threshold, such users may be required to satisfy training plan requirements. In some implementations, where compliance falls below a target threshold, an incentive plan may be implemented such that users will be incentivized to abide by the building facility's recommended health and safety practices. In some implementations, incentives could include food, prizes, additional vacation time, monetary bonus, and more. In some implementations, display 104 of dispensing station 101 may show the user profile and may provide advisory notices to maintain and/or improve compliance with the building or facility's recommended health and safety practices. In some implementations, analytics of dispensing station 101 at different locations may be used to determine a sanitization level and used to incentivize use of dispensing station 101 at specific location.

In some implementations, marketing plan may entail displaying statistics and/or benefits of recommended practices of sanitizing hands and/or washing hands on display 104 of dispensing station 101. In some implementations, display 104 may show images and/or videos for advertising products and/or businesses both within and outside the building or facility. In some implementations, dispensing station 101 may be utilized as an ambassador of products, services, businesses, and more. In some implementations, strategy may include integrated, customized advertising. In some implementations, marketing plans and/or advertisement programs may be shown at dispensing station 101 located where there is high foot traffic and ingress and egress of people either using or passing by the stations.

In some implementations contemplated in the present disclosure, determining a compliance based on analytics and calculations may be used to obtain better rates on medical health insurance, reduce sick-leave costs, and more. The present disclosure contemplates different levels of packages for monitoring automatic dispensing device 106. In some implementations, an essential package may provide only compiled data. In some implementations, a business package many include integrated analytics of compiled data, partnerships, and advertisements. In some implementations, an enterprise package may include premiere listing in advertisements, full analytics, marketing and advertising, as well as messaging packages to determine and analyze strategy plans, and more.

In some implementations, the present disclosure contemplates the monitoring system further includes an ultraviolet (UV) light sanitizer for sanitizing a personal object. In some implementations, dispensing station 101 includes a UV light sanitizer for sanitizing a personal object, such as a mobile phone, keys, and other personal objects. In some implementations, dispensing station 101 has an antimicrobial coated surface. In some implementations, the antimicrobial properties of coated surface may be activated by a UV light catalyst or a visible light spectrum catalyst.

In some implementations, the present disclosure contemplates dispensing station 101 has an interchangeable upper panel. In some implementations, the interchangeable upper panel may be a solar panel. In some implementations, the interchangeable upper panel may be shelving. In some implementations, the present disclosure contemplates dispensing station 101 structure may be adapted for endcap structure. In some implementations, positioning dispensing station 101 at an endcap may capitalize upon endcap profitability.

The present disclosure further contemplates the monitoring system may be adapted to various buildings and facilities, including office buildings, manufacturing facilities, retail businesses, universities, communal laundry facilities, gyms, and many more.

From the above description, it is manifest that various techniques can be used for implementing the concepts described in the present application without departing from the scope of those concepts. Moreover, while the concepts have been described with specific reference to certain implementations, a person having ordinary skill in the art would recognize that changes can be made in form and detail without departing from the scope of those concepts. As such, the described implementations are to be considered in all respects as illustrative and not restrictive. It should also be understood that the present application is not limited to the particular implementations described above, but many rearrangements, modifications, and substitutions are possible without departing from the scope of the present disclosure.

What is claimed is:

1. A monitoring system for use with an automatic dispensing device comprising:
at least one sensor;
a computing device, wherein the computing device includes a non-transitory memory storing an executable code; and
a hardware processor executing the executable code to:
receive an input from the at least one sensor, the input including a vibration information;
determine an occurrence of an event; and
update a station profile based on a signal that includes a station information.

2. The monitoring system of claim 1, further comprising:
a fluid reservoir storing a fluid for dispensing; and
a fluid dispensing mechanism fluidically connected to the fluid reservoir, the automatic dispensing device configured to dispense a user portion of the fluid when operated by a user, wherein the user portion of the fluid is a calibrated volume.

3. The monitoring system of claim 1, wherein the at least one sensor includes one or more of an accelerometer, a gyroscope, a magnetometer, a radiofrequency sensor, a vibration sensor, a motion sensor, a biometric sensor, a temperature sensor, a proximity sensor, an air quality sensor, a volatile organic compound sensor, and an image sensor.

4. The monitoring system of claim 1, wherein the station profile includes at least one of a refill identifier, a refill volume, a fill level information, a viscosity information, a date of last refill, a number of operations since last refill, a frequency of use information, a user counter, a battery level, and an air quality information.

5. The monitoring system of claim 1, wherein the non-transitory memory additionally stores a database including a plurality of user profiles and the monitoring system further comprises a scanner device, wherein the hardware processor further executes the executable code to:
receive an identification signal from the scanner device; and
update a first user profile of the plurality of user profiles based on the identification signal.

6. The monitoring system of claim 5, wherein the first user profile includes at least one of an individual identification, a time tracking information, a temperature reading, and a usage counter.

7. The monitoring system of claim 6, wherein the monitoring system is further configured to:
determine a compliance; and
formulate a strategy based on determining the compliance, wherein the strategy is implementing at least one of a training plan, a marketing plan, an incentive plan, and an advertisement program.

8. The monitoring system of claim 1, wherein the monitoring system is at least one of a battery-powered and a solar-powered.

9. The monitoring system of claim 1, wherein the monitoring system further includes an ultraviolet (UV) light sanitizer for sanitizing a personal object.

10. The monitoring system of claim 1, wherein the event is one of an operation of the automatic dispensing device and an individual walking past the automatic dispensing device.

11. A method for use with a monitoring system including a non-transitory memory and a hardware processor, the method comprising:
receiving, using the hardware processor, an input from at least one sensor, the input including a vibration information;
determining, using the hardware processor, an occurrence of an event; and
updating, using the hardware processor, a station profile based on a signal that includes a station information.

12. The method of claim 11, wherein the monitoring system monitors an automatic dispensing device having a fluid reservoir that stores a fluid for dispensing and a fluid dispensing mechanism fluidically connected to the fluid reservoir, wherein the automatic dispensing device is configured to dispense a user portion of the fluid, wherein the user portion of the fluid is a calibrated volume, and wherein the method further comprises:
dispensing a user portion when operated by a user.

13. The method of claim 11, wherein the at least one sensor includes one or more of an accelerometer, a gyroscope, a magnetometer, a radiofrequency sensor, a vibration sensor, a motion sensor, a biometric sensor, a temperature sensor, a proximity sensor, an air quality sensor, a volatile organic compound sensor, and an image sensor.

14. The method of claim 11, wherein the station profile includes at least one of a refill identifier, a refill volume, a fill level information, a viscosity information, a date of last refill, a number of operations since last refill, a frequency of use information, a user counter, a battery level, and an air quality information.

15. The method of claim 11, wherein the non-transitory memory additionally stores a database including a plurality of user profiles wherein the method further comprises:
receiving an identification signal from a scanner device; and
updating a first user profile of the plurality of user profiles based on the identification signal.

16. The method of claim 15, wherein the first user profile includes at least one of an individual identification, a time tracking information, a temperature reading, and a usage counter.

17. The method of claim 16, wherein the method further comprises:
determining a compliance; and
formulating a strategy, based on determining the compliance, wherein the strategy is implementing at least one of a training plan, a marketing plan, an incentive plan, and an advertisement program.

18. The method of claim 11, wherein the monitoring system is at least one of a battery-powered and a solar-powered.

19. The method of claim 11, wherein the monitoring system further includes an ultraviolet (UV) light sanitizer for sanitizing a personal object.

20. The method of claim 11, wherein the event is one of an operation of an automatic dispensing device and an individual walking past the automatic dispensing device.
